(19) United States
(12) Patent Application Publication
(10) Pub. No.: US 2025/0146044 A1
(43) Pub. Date: May 8, 2025

(54) RINSE SOLUTION FOR MICROBIAL DETECTION SYSTEMS

(71) Applicant: RAPID MICRO BIOSYSTEMS, INC., Lowell, MA (US)

(72) Inventors: Owen GRIFFIN, Boston, MA (US); Juan Pablo RODRIGUEZ SANTANA, Lowell, MA (US); Courtney RUSSELL, Hudson, NH (US); David JONES, Carlisle, MA (US)

(21) Appl. No.: 18/936,099

(22) Filed: Nov. 4, 2024

Related U.S. Application Data
(60) Provisional application No. 63/596,398, filed on Nov. 6, 2023.

(51) Int. Cl. C12Q 1/04

(52) U.S. Cl. CPC C12Q 1/04

(57) ABSTRACT

Exemplary embodiments pertain to a solution containing carbon black. The solution may be applied to a membrane filter of a microbial enumeration system as a post-rinse solution (i.e., after the membrane filter is rinsed with the sample of interest). The solution may reduce or suppress background autofluorescence, thereby improving detection and enumeration of colony forming units (CFUs) or other microbiological entities. The solution may be used in connection with a method for performing sample preparation for a microbial enumeration system. The carbon black solution may be manufactured according to a manufacturing process detailed herein.

### CROSS-REFERENCE TO RELATED APPLICATIONS

This application claims priority to and the benefit of U.S. Provisional Application No. 63/596,398, filed Nov. 6, 2023, the entirety of which is incorporated herein by reference.

### BACKGROUND

Many industries require the determination of the number or presence of microbes in a sample, often referred to as microbe enumeration or detection. One method of determining the number of microbes in a sample (or detecting the presence of microbes) involves exposing a filtration membrane to a sample to capture microbes in the sample on the membrane, culturing the microbes captured on the membrane, and optionally counting the number of colonies that grow during culturing.

Thus, membrane filtration is a commonly used method to concentrate and enumerate microorganisms present in a liquid sample. It allows for the filtration of a known volume of the sample through a membrane filter with a defined pore size, retaining microorganisms on the filter surface.

First, the liquid sample to be analyzed is typically diluted to reduce the microbial load and ensure the presence of single, isolated colonies on the filter. A membrane filter with an appropriate pore size is selected based on the expected size of the microorganisms being enumerated. Commonly used pore sizes are 0.45 μm or 0.2 μm.

The membrane filter is placed on a filter holder or manifold designed for microbial analysis. The filter holder is connected to a vacuum source, creating a pressure differential across the membrane. A known volume of the diluted sample is aseptically poured or pipetted onto the membrane filter, which is then placed on the filter holder. The vacuum is applied, and the liquid portion of the sample is drawn through the filter, leaving behind the microorganisms on the filter surface.

To ensure efficient recovery of microorganisms, the filter is rinsed or washed with an appropriate solution (such as sterile buffered saline) to remove any remaining sample residues or contaminants.

After filtration, the membrane filter containing the retained microorganisms is aseptically transferred to a suitable culture medium, such as agar plates or broth. The choice of medium depends on the specific requirements of the microorganisms being enumerated. The culture media containing the membrane filters are incubated under suitable conditions (temperature, time, and atmospheric conditions) to promote the growth of viable microorganisms. The incubation period is determined based on the expected growth characteristics of the target microorganisms.

After the incubation period, the membrane filters are examined, and visible microbial colonies that have grown on the filter surface are counted. The colonies are distinguished based on their characteristics, such as size, shape, color, and morphology. The colony counts obtained from the membrane filters are used to calculate the number of viable microorganisms in the original sample, taking into account the dilution factor and the volume of the filtered sample.

Membrane filtration is particularly useful when the sample volume is large, and the microbial load is low. It allows for concentrating microorganisms on the filter, enabling easier detection and enumeration of colonies compared to direct plating methods. The method is widely employed in various industries, including pharmaceuticals, food and beverage, water quality testing, and environmental monitoring.

Most conventional microorganism enumeration/detection systems use a visual detection method for enumerating microorganisms or colony forming units (CFUs) visible to the human eye. More recently, technology has been developed using a different enumeration/detection paradigm. In such systems, illuminated samples are caused to fluoresce without destroying the samples. By combining digital imagining technology with sophisticated software algorithms, these systems can detect and count the autofluorescence of growing microbes. An example of such a system is the GROWTH DIRECT™ system provided by RAPID MICRO BIOSYSTEMS™ of Lowell, Massachusetts.

To cause microorganisms to fluoresce, the membrane holding the sample is exposed to light, such as from one or more light emitting diode (LED) lamps, as shown in FIG. 1. This causes light to be emitted from the microorganisms, which is captured by photosensitive pixels on a charge-coupled device (CCD) chip.

However, some sample matrices that support the growth of CFUs may themselves have fluorescent properties, which may contribute background autofluorescence. Because conventional systems rely on visual detection, background autofluorescence is not generally a problem and conventional approaches typically do not take action to reduce autofluorescence. However, in a fluorescence-based approach the background fluorescence creates noise that can drown out the signal from the fluorescing CFUs, which can lead to inaccurate CFU counts.

Some techniques for addressing background autofluorescence that might be attempted are to dilute the sample, clarify the sample through the use of clarification filters, or treat the sample (e.g., by applying a dye). All of these possibilities have drawbacks, however. For example, dilution may not be possible due to regulatory sample-testing specifications and reporting requirements. Meanwhile, clarification filters and sample treatments may result in the removal of the organisms of interest, or mask contamination in the sample.

### BRIEF SUMMARY

Exemplary embodiments pertain to a solution containing carbon black. The solution may be applied to a membrane filter of a microbial enumeration system as a carbon black post-sample rinse solution (i.e., after the membrane is filtered with the sample and/or rinsed as discussed above). The solution may reduce or suppress background autofluorescence, thereby improving detection and enumeration of colony forming units (CFUs) or other microbiological entities.

In one aspect, a solution for a microbiological enumeration system includes carbon black, an emulsifier, and water. The emulsifier may be, for example, polysorbate 80, and the water may be deionized water. The emulsifier may be present in the solution at a concentration of about 1%.

Below a certain minimum concentration, the carbon black may be insufficient to effectively block background fluorescence, so that it remains difficult to perform enumeration (potentially resulting in false positives). Above a certain maximum concentration, the carbon black may obscure the fluorescence of the CFUs, thus reducing the signal received by the enumeration equipment and resulting in false negatives. According to exemplary embodiments, the carbon black may be present in the solution at a concentration of 0.0001%-0.5%.

The solution may be used in connection with a method for performing sample preparation for a microbial enumeration system. The method may include adding a growth medium to a sample analysis container (which may be, e.g., a petri plate or a prepared growth medium cassette/petri plate combination), filtering a sample to be tested through a filter membrane, applying the carbon black solution to the filter membrane as a rinse solution (which may be applied during a conventional rinse process or during a post-rinse process), and optionally placing the filter membrane on a growth medium.

The carbon black post-sample rinse solution may be manufactured according to a manufacturing process. The process may include mixing carbon black, an emulsifier, and water in a bulk solution mixer to form a mixed solution, adding the mixed solution to a container (e.g., a gas or plastic container), capping the container, and sterilizing the mixed solution. Capping the container may include applying a screw-top cap with a septum to the container.

The described embodiments may find application in several different areas of microbial enumeration, such as sterility testing and bioburden testing.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 depicts an example of a system that detects CFUs using fluorescence in accordance with one embodiment.

FIG. 2 is a flowchart depicting an exemplary method for deploying a carbon black post-sample rinse solution in accordance with one embodiment.

FIG. 3A depicts a simplified architecture for filling a cassette in accordance with one embodiment.

FIG. 3B depicts an exemplary cassette suitable for use with one embodiment.

FIG. 3C depicts an exemplary dual pressure valve in accordance with one embodiment.

FIG. 4 is a flowchart illustrating a method 400 for processing a sample for purposes of microbe enumeration or detection in accordance with one embodiment.

FIG. 5 is an example of a microbe enumeration or detection system in accordance with one embodiment.

FIG. 6 illustrates a method 600, which can be implemented to capture microbes from a samples for purposes of microbe enumeration or detection in accordance with one embodiment.

FIG. 7 illustrates a microbe enumeration system 700 including multiple cassettes in accordance with one embodiment.

FIG. 8 illustrates microbe enumeration or detection system including three cassettes, where each of the cassettes is coupled to a reservoir via a tube set.

FIG. 9 illustrates a microbe enumeration or detection system that may be provided in accordance with exemplary embodiments.

FIG. 10 depicts a technique for manufacturing a carbon black post-sample rinse solution in accordance with one embodiment.

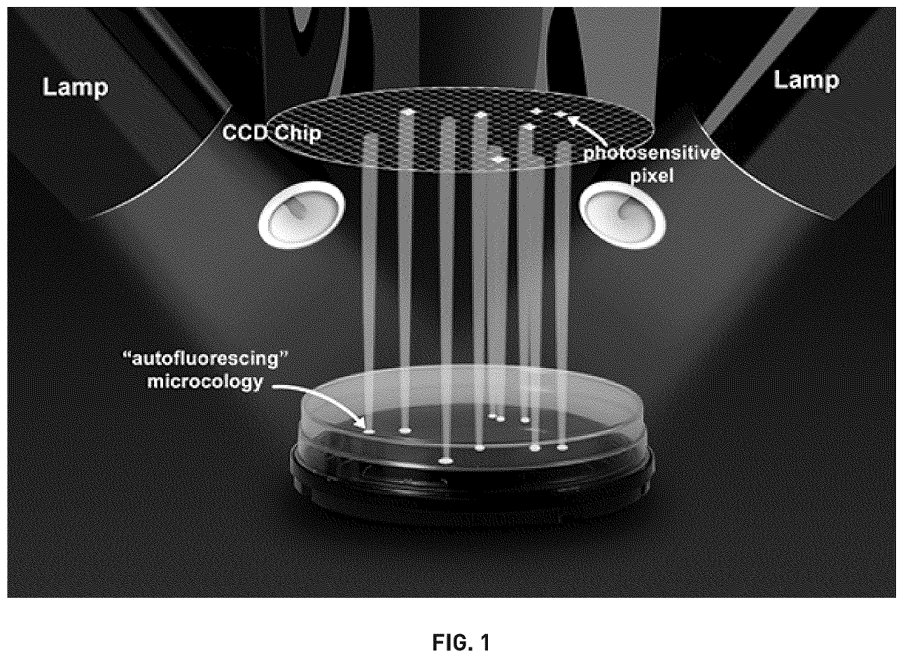

### DETAILED DESCRIPTION

Exemplary embodiments pertain to a Carbon Black Post-Sample Rinse Solution (CBPSRS) useful for lowering background fluorescence in a microbial enumeration and/or detection system. The microbial enumeration/detection system may be used to enumerate or detect colony forming units or other microbes in a sample that is typically tested in a cassette. The background fluorescence may be due to the sample, or to other materials on the surface of the applicable membrane (e.g., from other rinse solutions applied to the membrane).

The CBPSRS may also be used to help improve microorganism detection even when the background fluorescence is above required mean pixel intensity (MPI) requirements. For example, bioburden membrane imperfections and age can result in higher fluorescence background. This can be caused by the exposure of the agar surface or natural variations in the membrane. The use of the CBPSRS could suppress the increased background fluorescence and extend the use of materials.

In one exemplary embodiment, the solution is a 100±10 mL solution that is used to rinse a filter membrane containing a sample prior to placing the filter membrane on a growth medium. The recipe for the CBPRS is listed in Table 1.

| Ingredient | Weight or Volume | |
|---|---|---|
| Carbon Black | 0.000001-5.0 | g |
| Emulsifier | 0.00-1.0 | mL |
| DI water | 94.00-99.99 | mL |
| Total | 100 +/− 10 | mL |

Carbon black includes a group of intensely black, finely divided forms of amorphous carbon. According to exemplary embodiments, the carbon black may have an average particle size of 3.7-10.3 microns, preferably 5.0-9.0 microns, more preferably 6.0-8.0 microns, and most preferably about 7.0 microns

One example of a carbon black suitable for use with exemplary embodiments is Carbon Black MONARCH™ 4750 provided by CABOT™ CORPORATION of Boston Massachusetts. Other equivalent types of carbon black may also be used. Suitable types of carbon black may also include various types of recovered carbon black (rCB) filler products, such as KLEAN CARBON™ provided by KLEAN INDUSTRIES™ of Vancouver, British Columbia, Canada.

The emulsifier assists in dispersing the carbon black through the water so that it can evenly coat the membrane. The emulsifier may also or alternatively be a surfactant. An example of an emulsifier suitable for use with exemplary embodiments is Polysorbate 80, though equivalent emulsifiers may also be used.

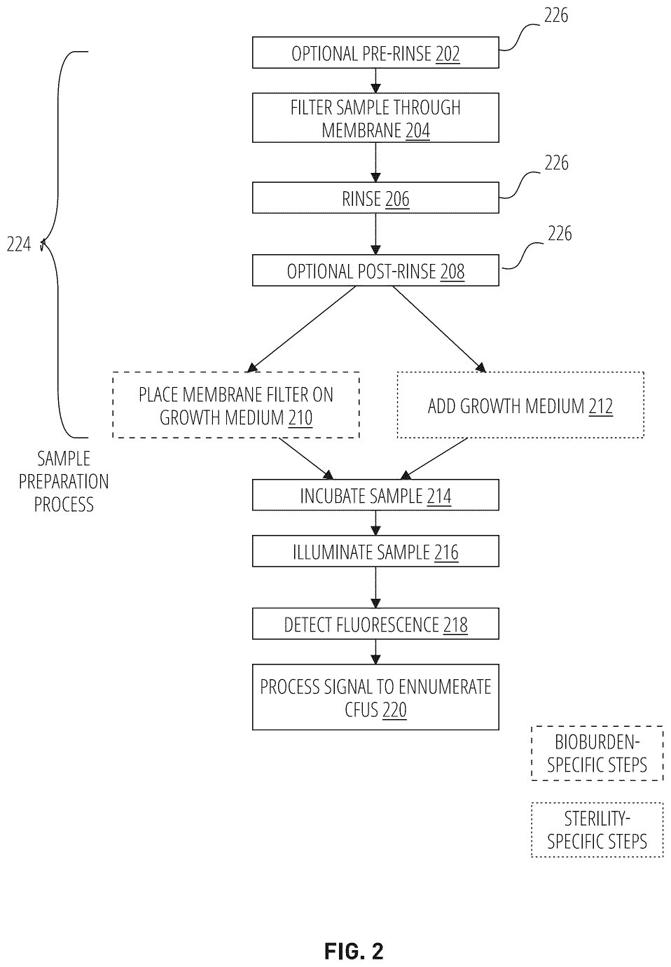

FIG. 2 depicts an exemplary microbe enumeration or detection process, during which the carbon black post-sample rinse solution may be applied.

Exemplary embodiments may be used with membrane filtration in different contexts, such as bioburden testing or sterility testing.

Bioburden testing is a process used to measure and quantify the number of viable microorganisms present on or in a product, component, or within an environment. It is an important quality control measure commonly used in industries such as pharmaceuticals, medical devices, cosmetics, and food and beverage.

The goal of bioburden testing is to assess the level of microbial contamination in a given sample. This information helps determine whether the product or environment meets the specified microbial limits or if further actions, such as sterilization or disinfection, are required to ensure product safety.

The bioburden refers to the total population of viable microorganisms, including bacteria, fungi, yeast, and molds. Bioburden testing typically involves sampling and analyzing the test samples using various methods such as agar plate count, membrane filtration, or direct microscopic counts. These methods allow for the enumeration and identification of different microorganisms present in the sample.

The testing is performed under controlled conditions using appropriate media and incubation parameters to support the growth of microorganisms. The results of bioburden testing provide critical data for assessing the effectiveness of manufacturing processes, validating sterilization methods, and monitoring the cleanliness and microbial control of controlled environments.

Bioburden testing is distinct from sterility testing. While bioburden testing assesses the total microbial load, sterility testing specifically focuses on the absence or presence of viable microorganisms, ensuring that a product or material is free from viable contaminants.

More specifically, Sterility testing is a critical quality control process used to determine the absence or presence of viable microorganisms in a product or material. It is commonly employed in industries such as pharmaceuticals, medical devices, biotechnology, and healthcare to ensure the sterility and safety of products intended for human use.

The purpose of sterility testing is to confirm that a product or material is free from viable microorganisms that could potentially cause infections or other adverse effects when administered or used. It is particularly important for sterile products, such as injectable drugs, implants, ophthalmic solutions, and surgical instruments.

Sterility testing is typically conducted using validated methods in specialized laboratories or cleanroom environments. The process involves taking a sample of the product or material and subjecting it to specific conditions that support the growth of any present microorganisms. The most commonly used method for sterility testing is the membrane filtration method.

In the membrane filtration method, the sample is passed through a sterile membrane filter with a defined pore size that can retain microorganisms. The filter is then transferred to a culture medium that supports the growth of microorganisms. The culture medium is incubated for a specified period, typically 14 days, to allow any viable microorganisms present in the sample to multiply and form visible colonies.

After the incubation period, the culture medium is examined for the presence of microbial growth. If no visible growth is observed, it indicates that the sample is considered sterile. However, if microbial growth is detected, it suggests the presence of viable microorganisms, indicating a failed sterility test and potential product contamination.

Sterility testing is performed in accordance with regulatory guidelines and standards to ensure accuracy, reliability, and reproducibility of the results. It is an essential part of the quality control process to verify the effectiveness of sterilization methods, validate manufacturing processes, and ensure the safety and efficacy of sterile products.

FIG. 2 depicts a general process that may be employed in bioburden or sterility testing. Bioburden-specific steps are indicated by dashed lines, while sterility-specific steps are indicated by dotted lines.

At block 204, a sample is prepared for analysis by filtration through a membrane (e.g., a 0.45 μm membrane filter). The membrane may then be rinsed with a rinse solution at block 206 (optionally, with a pre-rinse solution applied at block 202 and/or a post-rinse solution applied at block 208). The rinse, pre-rinse, and/or post-rinse assists with microbial recovery.

Exemplary embodiments may apply the carbon-black post-sample rinse solution as part of, or as, the pre-rinse, rinse, and/or post-rinse solution, or the carbon black post-sample rinse solution may be applied to the membrane filter following the application of the pre-rinse, rinse, and/or post-rinse solution(s).

In bioburden applications, after the carbon black post-sample rinse solution is applied, the membrane is placed on a growth medium (e.g., agar) in a cassette (block 212). In sterility applications, the membrane may be transferred to a media plate in block 212 and a growth medium (e.g., agar) may be added to a sample analysis container, such as a petri plate. Alternatively, the sample analysis container may be a cassette/petri plate that already includes a prepared growth medium.

Blocks 202-212 form a sample preparation process 224.

The cassette is then closed and the sample is permitted to incubate for a period of time (block 214). At the end of the incubation time, the sample is placed under one or more lamps (e.g., blue LED lamps), which illuminate the sample (block 216) and cause any microorganisms to fluoresce. The fluorescence may be detected by photosensitive pixels of a CCD device (block 218), and the resulting signal may be processed to detect and/or enumerate CFUs or other microbes in the sample (block 220).

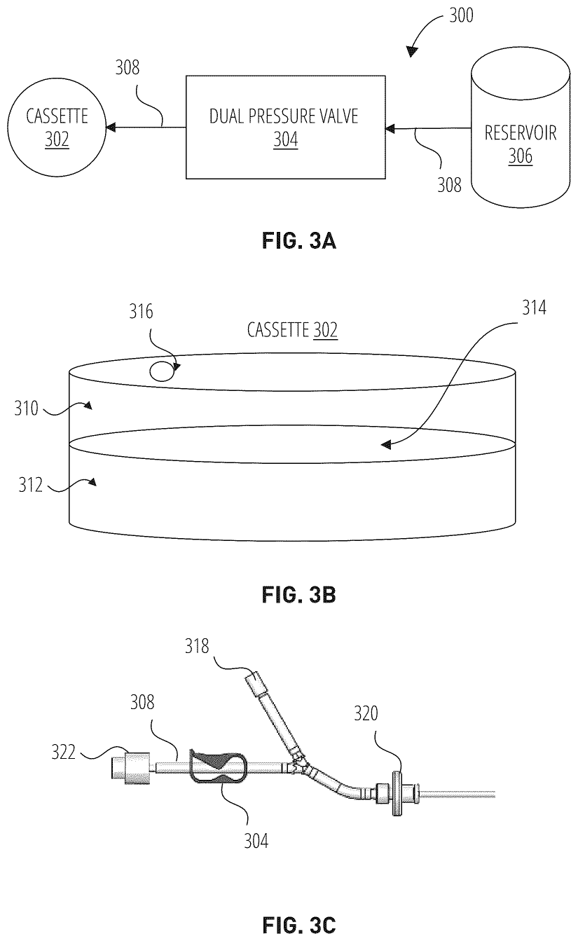

For context, FIGS. 3A-9 describe one example of a sample preparation process in more detail. The described preparation process is intended to be illustrative only, and one of ordinary skill in the art will recognize that other sample preparation techniques, including manual sample preparation techniques, may also be used with exemplary embodiments.

During microbe enumeration, a membrane (e.g., in a cassette, or the like) is exposed to a sample such that any microbes in the sample are captured on the membrane. One challenge to microbe enumeration is controlling distribution of the sample across the membrane.

Some systems use a vacuum to “draw” the sample into a chamber and across the membrane. One such vacuum system uses an open funnel containing the sample, which can introduce non-sample contaminants, thereby invalidating the enumeration process. Further, such system require that the membrane be kept under tension and further that a space is provided for the membrane to expand into.

The embodiments shown in FIGS. 3A-9 provide an enclosed microbe enumeration kit of a different type to provide a more consistent microbe enumeration process. In particular, the depicted embodiment provides a system including a number of cassettes, where each cassette includes a membrane. The cassettes are fluidly coupled to an enclosed reservoir by a tube set. The tube set includes a dual state pressure valve arranged to provide that the membrane is maintained in a relatively “flat” state during exposure to the sample. Maintaining the membrane in a relatively flat state ensures that the entirety of the membrane is exposed to (or wet by) the sample. Additionally, the dual state pressure valve is arranged to ensure that rinse fluid is properly filtered through the membrane and that the sample remains in an intended chamber of the cassette during exposure of the membrane to the sample.

FIG. 3A illustrates a microbe enumeration system 300, that may be provided in accordance with embodiments of the present disclosure. The microbe enumeration system 300 includes a cassette 302, a dual pressure valve 304, and a reservoir 306 fluidly coupled by a tube set 308. In general, cassette 302 can be any of a variety of containers or apparatuses arranged to provide for growth of microbes while reservoir 306 is a fluid container arranged to hold rinsing fluid, a sample containing microbes, or the like. During use, fluid (not shown) from the reservoir 306 flows into the cassette 302 via tube set 308 while the dual pressure valve 304 maintains pressure in the cassette 302 at one of two pressure states to ensure that the cassette 302 fills with fluid from reservoir 306 in an intended fashion. This is explained in greater detail below.

FIG. 3B illustrates cassette 302 in greater detail. As depicted in this figure, cassette 302 includes and upper chamber 310 and a lower chamber 312 with a membrane 314 disposed between the chambers. One of the upper chamber 310 or lower chamber 312 can be filled with microbe growth media (e.g., nutrient broth, lysogeny broth medium, or the like). Further cassette 302 includes at least one port 316 with which tube set 308 can couple to allow fluid from reservoir 306 to flow into cassette 302.

In some embodiments, membrane 314 can be a filter arranged to capture microbes from a sample of fluid in the reservoir 306. The operation of membrane 314 may require a certain pressure to both ensure that the membrane remains tight (e.g., does not vibrate, or tear) and also is sufficiently covered by the fluid. As example process to capture microbes on membrane 314 is described below.

FIG. 3C illustrates dual pressure valve 304 in greater detail. As depicted in this figure, dual pressure valve 304 includes a manually operated clamp which when engaged in one state compresses the tube set 308 to cause the pressure on one side of the tube set 308 to be higher and when engaged in the other state allows the tube set 308 to expand reducing the pressure in the one side of the tube set 308. With some example, tube set 308 can have a vent 318 arranged to stabilize the pressure in the tube set 308 on one side of the dual pressure valve 304 to atmosphere while the pressure in the tube set 308 on the other side of the dual pressure valve 304 is maintained at a higher pressure. Additionally, tube set 308 can include a flow restricter 320 arranged to restrict a fluid flow to below a certain pressure as well as a coupler 322 arranged to couple to port 316.

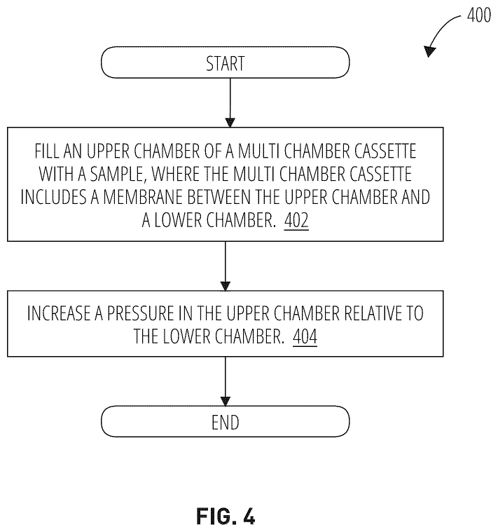

FIG. 4 illustrates a method 400 for processing a sample for purposes of microbe enumeration. Method 400 can be used with microbe enumeration system 300, or another system like the microbe enumeration system 300. In general, method 400 can be implemented to ensure that a membrane or filter (e.g., membrane 314 of microbe enumeration system 300) is sufficiently covered by a sample to capture microbes from the sample for purposes of microbe growth and enumeration. Method 400 can begin at block 400. At block 402 “fill an upper chamber of a multi chamber cassette with a sample, where the multi chamber cassette includes a membrane between the upper chamber and a lower chamber” upper chamber 310 is filled with fluid from reservoir 306. For example, reservoir 306 can be moved such that gravity causes fluid to flow from reservoir 306 through tube set 308 to upper chamber 310. With some examples, reservoir 306 can include one-way valves to allow air into reservoir 306 so that fluid can flow out. With a specific example, the reservoir 306 can include a one-way valve with a filter to inhibit the fluid in reservoir 306 from being contaminated with microbes from the air.

Continuing to block 404 “increase a pressure in the upper chamber relative to the lower chamber” pressure in upper chamber 310 can be increased relative to lower chamber 312 to cause fluid to sufficiently cover the membrane 314 and also to flow through the membrane 314 such that microbes in the fluid are captured by the membrane 314.

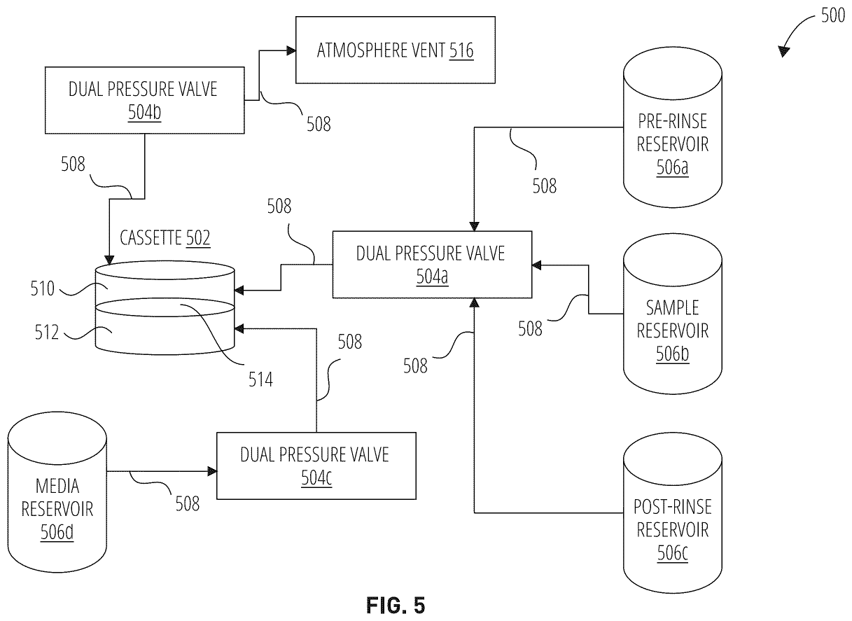

FIG. 5 illustrates a microbe enumeration system 500, that be provided in accordance with embodiments of the present disclosure. The microbe enumeration system 300 includes a cassette 502, dual pressure valves 504a, 504b, and 504c, and reservoirs 506a, 506b, 506c, and 506d fluidly coupled by a tube set 508. A number of different reservoirs are provided, such as, a pre-rinse reservoir 506a, sample reservoir 506b, post-rinse reservoir 506c, and media reservoir 506d. Sample reservoir 506b can contain a “sample” liquid medium, or said differently, a liquid medium to be analyzed with microbe enumeration as contemplated herein. Media reservoir 506d can include a liquid microbe growth medium. Microbe enumeration system 500 further includes an atmosphere vent 516 coupled to upper chamber 510. With some embodiments, microbe enumeration system 500 can include a fluid pump arranged to pump fluid in tube sets 508. However, for purposes of clarity, the fluid pump is not depicted in this figure.

During operation, fluid from reservoirs 506a, 506b, 506c, and 506d can flow (e.g., via pump action, via gravity, or the like) from the reservoirs into the cassette 502. Additionally, the dual pressure valves 504a, 504b, and 504c can be actuated to cause the pressure in upper chamber 510 to change relative to the pressure in lower chamber 512, or vice versa.

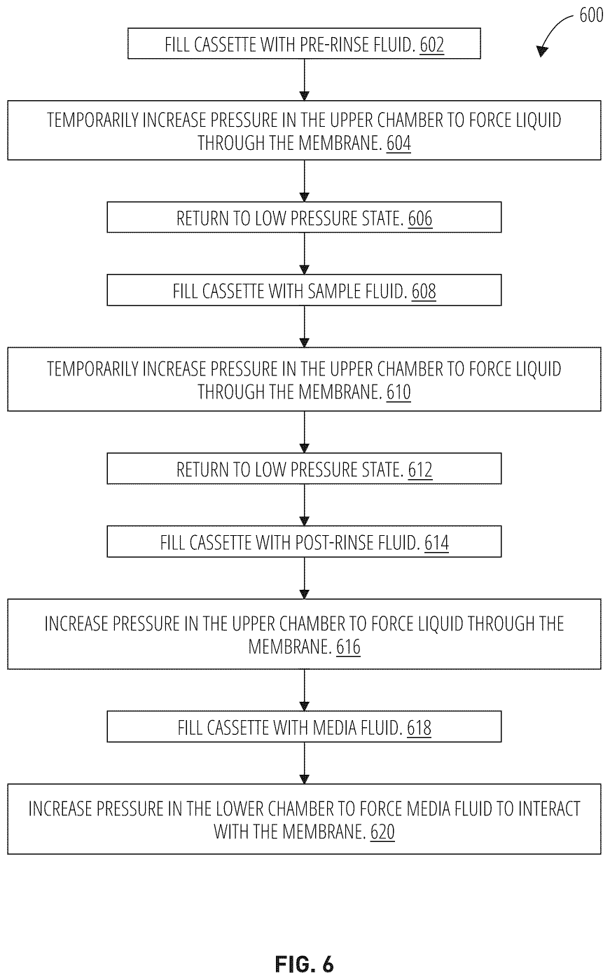

FIG. 6 illustrates a method 600, which can be implemented to capture microbes from a samples for purposes of microbe enumeration. Method 600 can be used with microbe enumeration system 500, or another system like the microbe enumeration system 500. In general, method 600 can be implemented to ensure that a membrane or filter (e.g., membrane 514 of cassette 502) is sufficiently covered by a sample (e.g., fluid from sample reservoir 506b) so as to capture microbes from the sample for purposes of microbe growth and enumeration.

Method 600 can begin at block 602. At block 602 “fill cassette with pre-rinse fluid” the cassette 502 can be filled with pre-rinse fluid from pre-rinse reservoir 506a. In particular, the upper chamber 510 of the cassette 502 can be filled with fluid from pre-rinse reservoir 506a during a period when the pressure in the upper chamber 510 is in a low state (e.g., less than or equal to 1.5 pounds per square inch (PSI) above atmosphere, between 0.5 and 1.5 PSI above atmosphere, or the like).

Continuing to block 604 “temporarily increase pressure in the upper chamber to force liquid through the membrane” the pressure in the upper chamber 510 can be temporarily increased to force fluid (e.g., the fluid added at block 602) in the cassette 502 through the membrane 514 and into the lower chamber 512. For example, the dual pressure valve 504b can be closed to pinch (or restrict) the tube set 508 between the upper chamber 510 and the atmosphere vent 516 to increase the pressure in the upper chamber 510. With some examples, closing the dual pressure valve 504b will increase the pressure between 0.5 and 4 PSI. With some embodiments, the pressure in the upper chamber can be increased for a set period of time (e.g., 30 seconds, 60 seconds, 90 seconds, or the like) while in other examples the pressure in the upper chamber can be increased until a condition is met (e.g., liquid flows through the membrane 514 into the lower chamber 512, or the like).

Continuing to block 606 “return cassette to low pressure state” the upper chamber 510 of cassette 502 can be returned to the low pressure state (e.g., less than or equal to atmosphere plus 1.5 PSI, or the like). Continuing to block 608 “fill cassette with sample fluid” the cassette 502 can be filled with sample fluid from sample reservoir 506b. In particular, the upper chamber 510 of the cassette 502 can be filled with fluid from sample reservoir 506b during a period when the pressure in the upper chamber 510 is in the low pressure state.

Continuing to block 610 “temporarily increase pressure in the upper chamber to force liquid through the membrane” the pressure in the upper chamber 510 can be temporarily increased to force fluid (e.g., the fluid added at block 608) in the cassette 502 through the membrane 514 and into the lower chamber 512. For example, the dual pressure valve 504b can be closed to pinch (or restrict) the tube set 508 between the upper chamber 510 and the atmosphere vent 516 to increase the pressure in the upper chamber 510. With some examples, closing the dual pressure valve 504b will increase the pressure between 0.5 and 4 PSI. With some embodiments, the pressure in the upper chamber can be increased for a set period of time (e.g., 30 seconds, 60 seconds, 90 seconds, or the like) while in other examples the pressure in the upper chamber can be increased until a condition is met (e.g., liquid flows through the membrane 514 into the lower chamber 512, or the like).

Continuing to block 612 “return cassette to low pressure state” the upper chamber 510 of cassette 502 can be returned to the low pressure state (e.g., less than or equal to atmosphere plus 1.5 PSI, or the like). Continuing to block 614 “fill cassette with post-rinse fluid” the cassette 502 can be filled with post-rinse fluid from post-rinse reservoir 506c. In particular, the upper chamber 510 of the cassette 502 can be filled with fluid from post-rinse reservoir 506c during a period when the pressure in the upper chamber 510 is in the low pressure state.

Continuing to block 616 “increase pressure in the upper chamber to force liquid through the membrane” the pressure in the upper chamber 510 can be increased to force fluid (e.g., the fluid added at block 614) in the cassette 502 through the membrane 514 and into the lower chamber 512. For example, the dual pressure valve 504b can be closed to pinch (or restrict) the tube set 508 between the upper chamber 510 and the atmosphere vent 516 to increase the pressure in the upper chamber 510. With some examples, closing the dual pressure valve 504b will increase the pressure between 0.5 and 4 PSI. With some embodiments, the pressure in the upper chamber can be increased for a set period of time (e.g., 30 seconds, 60 seconds, 90 seconds, or the like) while in other examples the pressure in the upper chamber can be increased until a condition is met (e.g., liquid flows through the membrane 514 into the lower chamber 512, or the like).

Continuing to block 618 “fill cassette with media fluid” the cassette 502 can be filled with media fluid from media reservoir 506d. In particular, the lower chamber 512 of the cassette 502 can be filled with fluid from media reservoir 506d during a period when the pressure in the upper chamber 510 is in the high pressure state.

Continuing to block 620 “increase pressure in the lower chamber to force media fluid to interact with the membrane” the pressure in the lower chamber 512 can be increased to force media fluid (e.g., the media fluid added at block 618) in the cassette 502 to interact with the membrane 514. For example, the dual pressure valve 504c can be closed to pinch (or restrict) the tube set 508 between the lower chamber 512 and the media reservoir 506d to increase the pressure in the lower chamber 512. Furthermore, the dual pressure valve 504a can be closed to pinch (or restrict) the tube set 508 between the other reservoirs and the upper chamber 510 to increase the pressure in the lower chamber 512. With some examples, the pressure in the lower chamber 512 can be raised between 0.5 and 4 PSI. With some embodiments, the pressure in the upper chamber can be increased for a set period of time (e.g., 30 seconds, 60 seconds, 90 seconds, or the like) while in other examples the pressure in the lower chamber can be increased until a condition is met (e.g., liquid stops moving through the tube set 508, or the like).

In some embodiments, method 600 can include a further step of disconnecting the cassette 502 from the tube set 508 and processing the cassette in a microbe culturing process (e.g., exposing to light, darkness, heat, moisture, or the like).

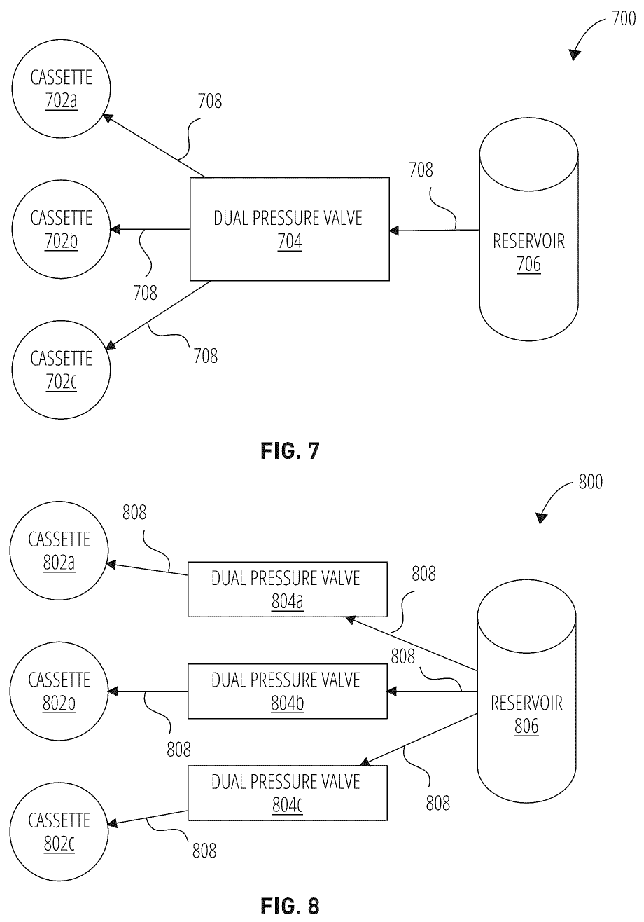

FIG. 7 illustrates a microbe enumeration system 700 including multiple cassettes. In particular microbe enumeration system 700 includes cassettes 702a, 702b, and 702c where each of the cassettes is coupled to reservoir 706 via tube set 708. Cassettes 702a, 702b, and 702c can be filled with fluid from reservoir 706 using a similar process as provided elsewhere herein (e.g., with respect to FIG. 4, FIG. 6, etc.). The tube set 708 is coupled (e.g., mechanically, or the like) to a single dual pressure valve 704, which can be utilized to effect a change in pressure in the cassettes 702a, 702b, and 702c as described elsewhere herein. Additionally, microbe enumeration system 700 can include multiple reservoirs 706 (e.g., like in microbe enumeration system 500 of FIG. 5, or the like).

In other examples, multiple dual pressure valves 704 can be provided. For example, FIG. 8 illustrates microbe enumeration system 800 including cassette 802a, 802b, and 802c where each of the cassettes is coupled to reservoir 806 via tube set 808. Cassettes 802a, 802b, and 802c can be filled with fluid from reservoir 806 using a similar process as provided elsewhere herein (e.g., with respect to FIG. 4, FIG. 6, etc.). The tube set 808 is coupled (e.g., mechanically, or the like) to multiple dual pressure valves. In particular, tube set 808 is coupled to dual pressure valves 804a, 804b, and 804c. In this manner, pressure in cassette 802a, 802b, and 802c can be controlled individually.

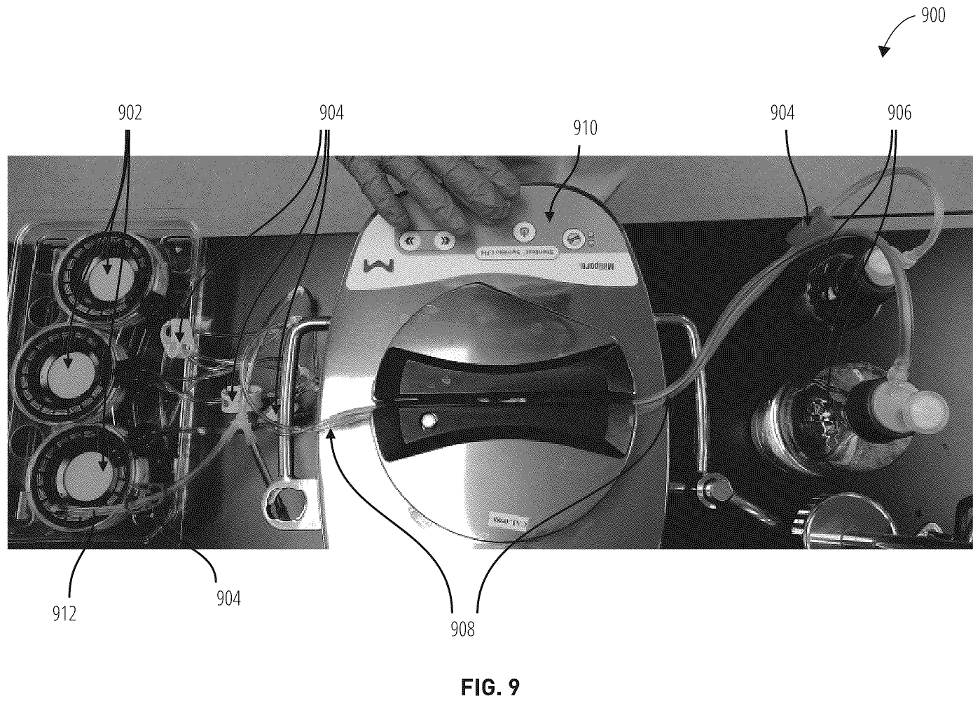

As indicated above, in some embodiments, the example microbe enumeration systems (e.g., microbe enumeration system 300, microbe enumeration system 500, microbe enumeration system 700, microbe enumeration system 800, or the like) can include a pump. FIG. 9 illustrates a microbe enumeration system 900 that may be provided in accordance with embodiments of the present disclosure. The microbe enumeration system 900 includes a cassettes 902, dual pressure valves 904, and reservoirs 906 fluidly coupled by tube set 908. Furthermore, a pump 910 is provided to assist in moving (e.g., via capillary action, or the like) fluid from reservoirs 906 to cassettes 902 via tube set 908. Additionally, an atmosphere vent 912 is provided. During operation, fluid can flow from the reservoirs 906 into the cassettes 902. Additionally, the dual pressure valves 904 can be actuated to cause the pressure in the upper and/or lower chambers of the cassettes 902 to be increased or decreased as described herein to assist in wetting the membranes in the cassettes 902 with fluid from the reservoirs 906.

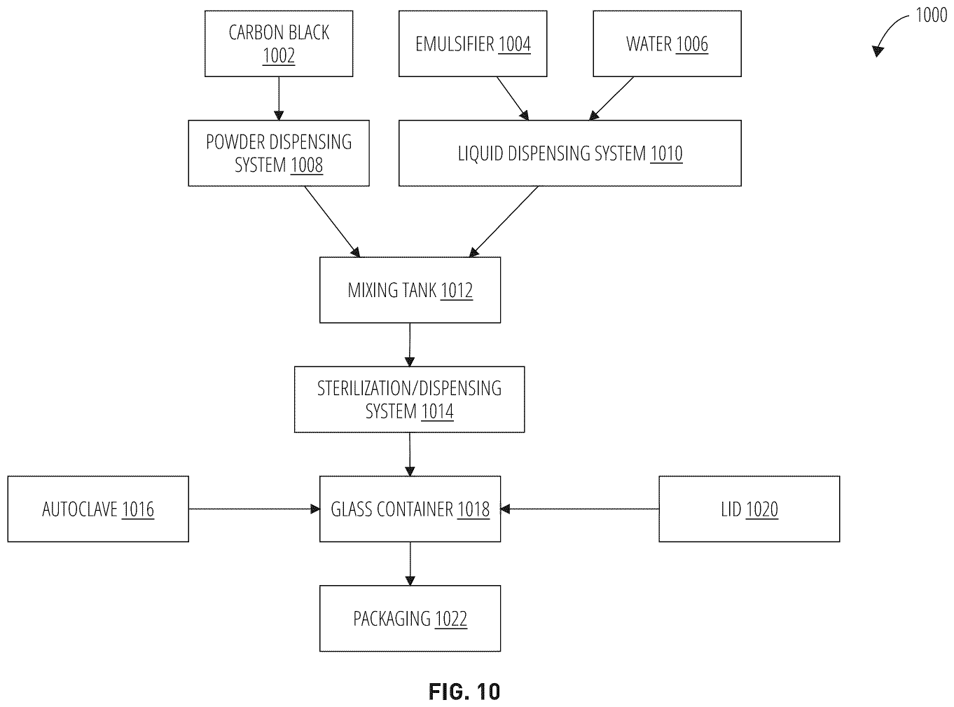

FIG. 10 depicts an exemplary manufacturing process suitable for producing a CBPRS.

Carbon black 1002, emulsifier 1004, and water 1006 may be mixed together in a mixing tank 1012, such as a stainless steel mixing tank. The carbon black 1002 may be added in an amount sufficient to result in a concentration, within the CBPRS, of 0.0001%-0.5% carbon black. The emulsifier 1004 may be polysorbate 80 or an equivalent, and may be added in an amount sufficient to result in a concentration, within the CBPRS, of 1% emulsifier. The remainder of the CBPRS may be water 1006 in the form of deionized water.

The carbon black 1002 may be in the form of a powder, and therefore may be dispensed into the mixing tank 1012 using a powder dispensing system 1008. This may be an automated powder dispensing system 1008; alternatively, the carbon black 1002 may be manually added to the mixing tank 1012.

Similarly, the liquid components (the emulsifier 1004 and the water 1006) may be dispensed into the mixing tank 1012 using a liquid dispensing system 1010, which may be an automated liquid dispensing system 1010. Alternatively, the liquids may be manually added to the mixing tank 1012.

The mixing tank 1012 may mix the components together using a standard bulk solution mixing process. The solution, during or after mixing, may be sterilized. In one embodiment, the mixed solution is transferred from the mixing tank 1012 to a sterilization/dispensing system 1014 to be sterilized and/or dispensed into glass or plastic (e.g., autoclavable plastic) containers 1018. The solution may be sterilized using a validated sterilization process.

The glass container 1018 may be sized to hold about 100 mL of the CBPRS. A lid 1020 may be secured to the glass container 1018. The lid 1020 may be, for example, a screw-on cap with a septum. When the CBPRS is used, the septum may be pierced by a needle or cannula and applied to the membrane filter, in order to preserve the sterility of the CBPRS. The assembled glass container 1018 may be sterilized using an autoclave 1016.

After the cap is applied and the assembly is autoclaved the glass container 1018 may be packaged in packaging 1022 until the time that it is used.

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in embodiments.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A solution for a microbiological enumeration or detection system, comprising:
carbon black;
an emulsifier; and
water.

2. The solution of claim 1, wherein the carbon black has an average particle size in the range of 6.0-8.0 microns.

3. The solution of claim 1, wherein the emulsifier is polysorbate 80.

4. The solution of claim 3, wherein the emulsifier is present in the solution at a concentration of about 1%.

5. The solution of claim 1, wherein the carbon black is present in the solution at a concentration of at least 0.0001%.

6. The solution of claim 1, wherein the carbon black is present in the solution at a concentration of at most 0.5%.

7. The solution of claim 1, wherein the solution is a post-sample rinse solution.

8. A method for performing sample preparation for a microbial enumeration or detection system, comprising:
adding a growth medium to a sample analysis container;
filtering the sample through a membrane;
filtering the solution of claim 1 through the membrane.

9. The method of claim 8, further comprising adding a growth medium to a sample analysis container.

10. The method of claim 8, further comprising filtering a rinse solution through the membrane.

11. The method of claim 8, wherein the sample analysis container is a cassette, and further comprising filling the cassette with growth media.

12. A method for manufacturing a carbon-black post-rinse solution, the method comprising:
mixing carbon black, an emulsifier, and water in a bulk solution mixer to form a mixed solution;
adding the mixed solution to a glass or plastic container;
capping the glass or plastic container; and
sterilizing the mixed solution.

13. The method of claim 12, wherein capping the container comprises applying a screw-top cap with a septum to the container.

14. The method of claim 12, wherein the emulsifier is polysorbate 80.

15. The method of claim 12, wherein the carbon black is present in the solution at a concentration of at least 0.025%.

16. The method of claim 12, wherein the carbon black is present in the solution at a concentration of at most 0.1%.
